(19) United States
(12) Patent Application Publication
(10) Pub. No.: US 2003/0235905 A1
(43) Pub. Date: Dec. 25, 2003

(54) Base member for a biochip

(75) Inventor: Heinrich Spiecker, Bielefeld (DE)

Correspondence Address: JORDAN AND HAMBURG LLP, 122 EAST 42ND STREET, SUITE 4000, NEW YORK, NY, 10168, US

(73) Assignee: LaVision BioTec GmbH, Bielefeld (DE)

(21) Appl. No.: 10/459,854

(22) Filed: Jun. 12, 2003

(30) Foreign Application Priority Data
Jun. 22, 2002 (DE) 102 27 962.4-52

(51) Int. Cl. C12M 1/34; C12M 3/00

(52) U.S. Cl. 435/287.1

(57) ABSTRACT

A base member for a biochip, e.g., a chip for analyzing DNA, RNA, proteins or cells, the base member (1), which is shaped like a board, being provided with a plurality of discrete elevations (2) extending from the upper side (1a) to the underside and having, in the region of their lower end, an active surface (3) for receiving the probe material.

### FIELD OF THE INVENTION

The invention relates to a base member for a biochip e.g., for a chip for analyzing DNA, RNA, proteins or cells.

### DESCRIPTION OF THE PRIOR ART

Biochips are known. Said biochips are members with a probe material uniformly distributed over a relatively small surface area thereof and applied thereon using e.g., a device similar to an inkjet printer. This probe material will react when coming into contact with a sample, the DNA or RNA to be analyzed for example. As the base member for a biochip is structured using a device operating in a way similar to that of an inkjet printer, there is always the risk that the points are not applied where they should. This means that when the biochip has come into contact with the sample material and the probe material reacts accordingly, the pattern will possibly not match the receiver optics pattern. This may lead to false measurements. False measurements may also be due to the following:

Sample material is placed onto the base member, meaning the biochip, provided with the probe material. After the reaction has taken place, the surface of the chip must be washed and cleaned to permit to see which probe material has reacted with the sample material. The steps of washing and cleaning are critical inasmuch as but specifically fixed sample material is allowed to remain on the chip. This however depends on various parameters such as temperature, salt concentration and the treatment the chip is otherwise subjected to.

### BRIEF SUMMARY OF THE INVENTION

It is therefore the object of the invention to provide a base member for a biochip which provides for easy attachment of the probes on the one side and which, on the other side, allows for fast and reliable evaluation of the sample.

In accordance with the invention, the solution to this object is to configure the base member as a board which is provided on the underside thereof with a plurality of discrete elevations extending from the upper side to the underside and having, in the region of their lower end, an active surface for receiving the probe material. As a result, to attach the probe material to the base member, it suffices to place the base member with the various elevations, which are for example in the shape of a truncated pyramid, into the corresponding recesses of a nanotiter plate, said nanotiter plate comprising individual wells containing different probe material. The active surface at the lower end of each elevation causes the probe material to be picked up and attached in a certain manner. This may be achieved in that the active surface from e.g., 50 to 200 ·m in diameter is activated using methods known from the literature (The Chipping Forecast, Nature Genetics Supplement, vol. 21, January 1999and DNA Microarrays: A Practical Approach, Mark Schenar, Oxford University Press, September 1999), said active surface picking up the probe material and fixating it.

According to a particularly advantageous feature of the invention there is provided that a lens is disposed locally above each elevation or above each truncated cone respectively. In disposing such type microlenses above each elevation, one achieves that the light is focussed by the front surface of the truncated cones or by the elevations respectively, meaning by the active surface, and that the radiation can be sensed by a detection optics. More specifically, the microlenses permit to image and magnify the widely spaced apart active surfaces of the various elevations or truncated cones closely next to each other on the detector. The arrangement of the lenses, and in the present case more specifically of the microlenses above the base member has the further advantage that, in irradiating the active surfaces of the chip with a light source, e.g., with white light or laser light, in order for example to generate a fluorescence radiation, the lenses focus the light in such a manner that it is concentrated and focused on the active surface. This permits to reduce possible stray radiation.

Another subject matter of the invention is a device for reading the chip according to one or several of the features discussed herein above, a radiation source and a detection optics being provided for reading fluorescence for example. The radiation source may hereby be a white light source, a laser or an array of LEDs or of laser diodes. Various excitation wavelengths and different emission filters may be used, which allows differential measurements to be performed.

Advantageously, an aperture plate is disposed between the radiation source and the biochip, said aperture plate permitting to shield from fractions of scattered light. The aperture plate arrangement is hereby advantageously disposed in the intermediate image plane produced by the microlenses of the chip. The detection optics preferably comprises a CCD camera, if necessary with filters set in front thereof for accordingly filtering the fluorescence emission.

The arrangement can also be used if chemoluminescence is used for reading the chip. The excitation optics, that is the radiation source and the beam splitter, may be dispensed with, though. Using chemoluminescence, the radiation source may more specifically be dispensed with because the emitted light is produced by the chemical reaction. This is also the reason why no filters are needed with chemoluminescence.

Another subject matter of the invention is a device for hybridizing the chip according to one or a plurality of the claims 1 to 5 with sample material; such a device is substantially characterized by a cup-shaped element for receiving the underside of the chip, said cup-shaped element receiving the sample material and being more specifically provided, in the bottom region thereof, with a membrane for covering the active surface of the elevations, which may also be termed nests, at certain time intervals by bringing them into contact with said membrane. Another possibility could be to configure the bottom in such a manner that it is adapted to tightly cover the active surface without the bottom being configured as a membrane. The bottom can be a membrane, though. As a result thereof, it is possible to make the reading process dynamical inasmuch as, in varying individual parameters such as temperature, salt content and so on, the reaction between the probe material and the sample material may be varied and this variation in the sample material makes it possible to perform a plurality of consecutive measurements by periodically covering the active surfaces. The membrane can hereby also be the bottom of the cup-shaped element. The cup-shaped element is covered by the base member acting as a substantially airtight cover. If, by means of fluidic connections, a negative pressure is applied to the trough, which is closed by the base member of the biochip, the membrane, or the membrane configured to form the bottom respectively, will come to rest against the active surface, thus displacing the sample fluid and covering the active surface as a result thereof. If now the chip is read using fluorescence, meaning if it is exposed to light radiation, the excitation light will no longer penetrate the actual sample fluid but will only reach the molecules of the probe material or of the sample material respectively which are fixed to the active surface. This prevents excitation and detection of background fluorescence from the very sample fluid. If the negative pressure is eliminated, the membrane moving away from the active surface as a result thereof, sample fluid comes again into contact with the active surface so that the reaction can be resumed and the measurement repeated. This makes it possible to dynamically keep track of the reaction between the probe material and the sample material. Using a membrane by way of bottom, or in addition to a bottom respectively, the sample fluid may also be so to speak circulated by periodically applying negative pressure, which possibly permits to reduce reaction times. For this purpose, it might be necessary to design the elevations or nests in such a manner that the periodical movement causes the fluid to circulate in one direction within the trough, thus leading to a faster and more efficient reaction. In the region between the elevations, the bottom of the trough may also be designed in such a manner that the volume between bottom and chip is kept small, e.g., by configuring the bottom as a nanotiter plate. This permits to work with a smaller sample volume which makes picking up of the samples and preparation less costly and more convenient.

The invention will be explained in greater detail herein after with reference to the drawings.

### BRIEF DESCRIPTION OF THE SEVERAL VIEWS OF THE DRAWING

FIG. 1 is a sectional side view of a portion of a biochip;

FIG. 2 shows the biochip disposed in a nanotiter plate;

FIG. 3 shows the chip disposed in a cup-shaped element with a bottom configured as a membrane;

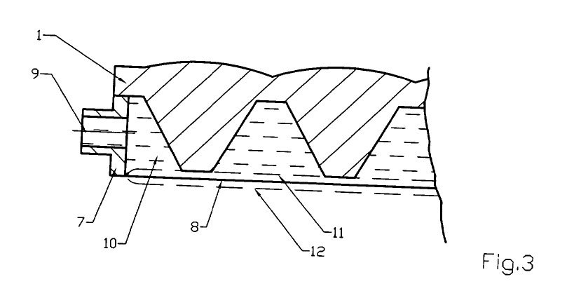

FIG. 4 shows the biochip in the cup-shaped element with the radiation optics and the reading optics.

### DETAILED DESCRIPTION OF THE INVENTION

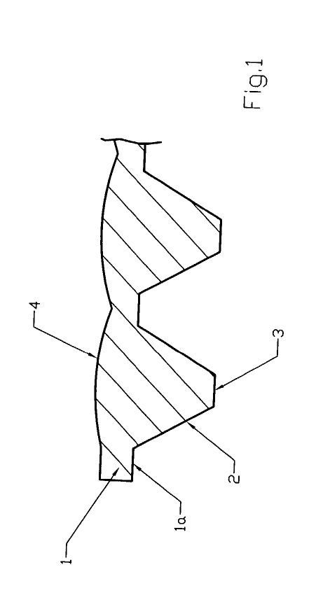

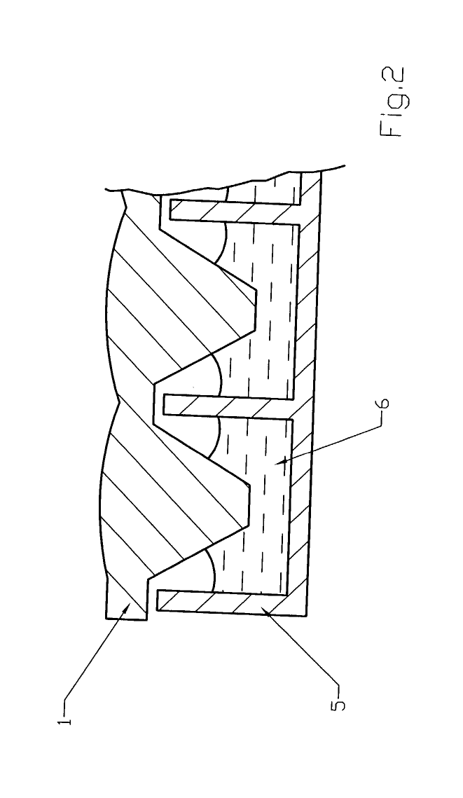

According to FIG. 1, the biochip indicated at 1 shows elevations 2 formed on its upper side la, said elevations being in the shape of a truncated cone. There is one microlens on top of each elevation. At the lower end of a respective one of the elevations 2 in the shape of a truncated cone there is what is termed an active surface 3 for picking up probe material from the various receptacles in the nanotiter plate 5 (see FIG. 2). As already explained, the nanotiter plate 5 shows various receptacles holding a different probe material each, said probe material serving to wet the active surface 3 of the biochip 1. The probe material is hereby attached to the active surface 3 in an appropriate manner so that, upon reaction with the sample material and adequate luminescence excitation, it emits the corresponding radiation. For hybridization with probe material, there is provided that, once the probe material is attached to the active surface 3, the biochip is immersed into a cup-shaped element 7, such as a trough for example, the active surfaces 3 coming into contact with the sample material 10.

As already explained herein above, there is provided, according to one feature of the invention, that the bottom 8 of the trough-shaped element 7 be configured as a membrane. Inasmuch, the bottom can adopt two different positions; in one of these positions, the bottom fits against the active surface 3 of the biochip, displacing the excess sample material (arrow 11). This is the case if a negative pressure is applied to the fluidic connection 9. In the case of excess pressure, by contrast, the membrane 8 adopts the position shown by arrow 12. In periodically applying negative or excess pressure onto the membrane 8, the sample material is well mixed which is particularly advantageous when measurements are to be performed almost continuously at the active surface of the biochip with the sample material varying every time.

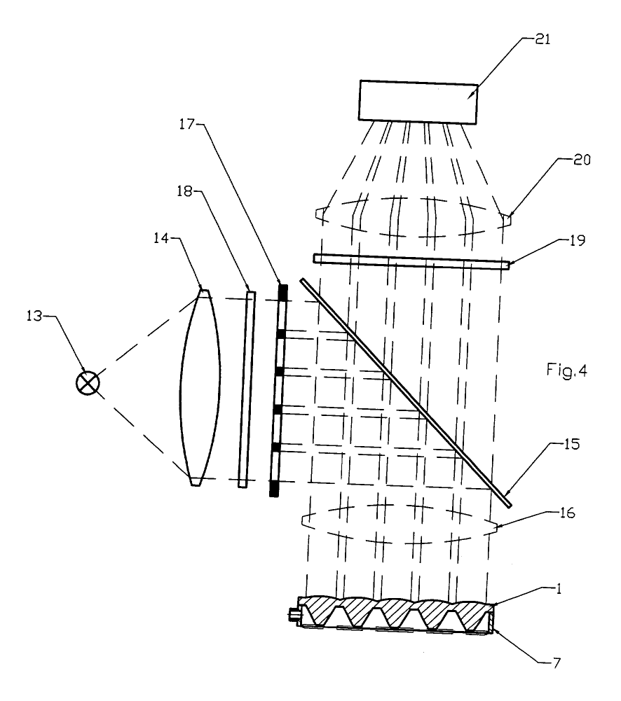

Thearrangement as it can be seen from FIG. 4 serves to read the biochip. A radiation source 13 is thereby provided which irradiates the active surfaces 3 of the biochip 1 through the microlenses 4, said radiation passing through the illumination optics 14 and the excitation filter 18, through the aperture plate 17 disposed downstream in the beam path and through the coupling mirror 5. The fractions of scattered light can be screened by the aperture plate 17 in particular. In the beam path there is a first imaging optics 16 located beneath the coupling mirror 15; downstream of said imaging optics 16 there is a second imaging optics 20 located behind the detection filter 19. The detector 21, a CCD camera for example, senses the luminescence emitted by each of the active surfaces of an elevation or nest of the biochip.

Listing of Numerals

1. biochip

2. elevations on the underside

3. active surface

4. microlens

5. nanotiter plate

6. probe material

7. cup-shaped element (trough)

8. bottom of the cup-shaped element, configured as a membrane

9. fluidic connection

10. hybridization volume

11. contacting position of the membrane when negative pressure prevails in the trough

12. position of the membrane when excess pressure prevails in the trough

13. radiation source

14. illumination optics

15. coupling mirror

16. imaging optics I

17. aperture plate

18. excitation filter

19. detection filter

20. imaging optics II

21. detection optics

I claim:

1. A base member for a biochip, e.g., a chip for analyzing DNA, RNA, proteins or cells,
characterized in that the base member (1), which is shaped like a board, is provided with a plurality of discrete elevations (2) extending from the upper side (1a) to the underside and having, in the region of their lower end, an active surface (3) for receiving the probe material.

2. The base member according to claim 1,
characterized in that the elevations (2) are configured in the shape of a truncated cone or pyramid.

3. The base member according to claim 1,
characterized in that the active surface (3) is between 50 and 200 ·m in diameter.

4. The base member according to claim 1,
characterized in that the elevations (2) are disposed in a pattern matching a nanotiter plate (5).

5. The base member according to claim 1,
characterized in that a lens (4) is disposed locally above each elevation (5).

6. An arrangement for reading the chip according to one or several of the claims 1 through 5,
characterized by a detection optics (21).

7. The arrangement according to claim 6,
characterized in that the arrangement comprises a radiation source (13).

8. The arrangement according to claim 7,
characterized in that the radiation source (13) is a white light source, a laser or an array of LEDs or laser diodes.

9. The arrangement according to claim 7,
characterized in that an aperture plate arrangement (17) for screening fractions of scattered light is provided between the radiation source (13) and the chip (1).

10. The arrangement according to claim 9,
characterized in that the aperture plate arrangement (17) is hereby disposed in the intermediate image plane produced by the microlenses (4).

11. The arrangement according to claim 6,
characterized in that the detection optics (21) comprises a CCD camera.

12. The arrangement according to claim 6,
characterized in that the detection optics (21) has a filter (19).

13. A device for hybridizing the chip with sample material according to one or several of the claims 1 through 5,
characterized by a cup-shaped element (7) for receiving the underside of the chip (1), said cup-shaped element (7) receiving the sample material (10) and said cup-shaped element (7) being adapted to preferably periodically cover the active surface (3) of the elevations (2) in the region of the bottom (8) thereof so that the reading can be configured to be a dynamical process by varying the parameters.

14. The device according to claim 12,
characterized in that the cup-shaped element (7) is tightly closed by the chip (1), an access (9) being provided for producing a negative pressure in the cup-shaped element (7).

15. The device according to claim 13,
characterized in that the bottom (8) is configured to be a membrane.
